(19) United States
(12) Patent Application Publication
(10) Pub. No.: US 2022/0192599 A1
(43) Pub. Date: Jun. 23, 2022

(54) PATIENT-WORN SENSOR INCLUDING COMPLIANT FLEXIBLE PRINTED CIRCUIT ASSEMBLY

(71) Applicant: MURATA VIOS, INC., Woodbury, MN (US)

(72) Inventors: Michael MARSHIK, Woodbury, MN (US); Brandon LA PLANTE, Woodbury, MN (US); Dattatraya BATHE, Woodbury, MN (US); Scott MAZAR, Woodbury, MN (US); Nobuhiro KONDO, Woodbury, MN (US); Atsuhiko HIRATA, Woodbury, MN (US)

(21) Appl. No.: 17/604,598

(22) PCT Filed: Apr. 28, 2020
(86) PCT No.: PCT/US2020/030177

§ 371 Date: Oct. 18, 2021

Related U.S. Application Data
(60) Provisional application No. 62/704,065, filed on Apr. 30, 2019.

(51) Int. Cl. A61B 5/00; A61B 5/25

(52) U.S. Cl. CPC A61B 5/6801; A61B 5/25; A61B 2560/0456; A61B 2562/166; A61B 2562/164

(57) ABSTRACT

A patient-monitoring device includes an adapter with a flexible printed circuit, the flexible printed circuit includes a rigid portion with a rigid stiffener and a flexible portion with a trace, and with a connector that is connected to one end of the trace, that is movable in two or three dimensions independent of the rigid portion, and that is connectible to an electrode assembly.

### BACKGROUND OF THE INVENTION

### 1. Field of the Invention

The present invention relates to a patient-worn sensor monitoring device that includes an electrode assembly that, in use, is temporarily affixed or adhered to the skin of a patient.

### 2. Description of the Related Art

Many different types of patient monitoring systems require a direct electrical interface to the skin of a patient. In some applications, the direct electrical interface to the patient's skin is to sense electrical signals present at that skin location; while in other applications, the direct electrical interface is to apply an electrical current stimulation signal at that skin location. Therefore, the patient monitoring systems typically require a patient-worn sensor assembly that detects, records, and communicates patient data. As such, the patient-worn sensor assembly can include several structural features that can provide increased signal quality, reduction in signal noise, increased patient comfort, increased reliability, and increased adhesion to a patient's skin.

The patient-worn sensor can receive vital-sign information, such as blood pressure, body temperature, respiratory rate, blood oxygenation, electrocardiogram (ECG), heart rhythm, heart rate, blood glucose level, and hydration (bio-impedance) levels, etc. The patient-worn sensor can also track and record additional information about patients, including patient movement, activity, and sleep patterns.

A conventional patient-worn sensor collects information sensed at the patient's skin and wirelessly transmits the data to another device of a monitoring system (e.g., bed-side monitor, tablet device, mobile phone, central processing server, etc.), which in turn can be connected to a network system of a hospital, clinic, or home-based monitoring system. Such a patient-worn sensor can include an adhesive electrode assembly with multiple individual electrodes that are attached to the patient's skin, and a sensor assembly that includes all of the sensing, processing and communication electronics, and a power supply in a self-contained sensor-transmitter device. In this conventional sensor-transmitter device, the electrode assembly provides a direct electrical interface, an adhesive to attach to the patient's skin, and a platform to which the sensor assembly connects and is supported.

A conventional skin electrode assembly is connected by an electrical lead (i.e., a long wire) to a monitoring system and is intended to be used with the patient “tethered” to a monitoring system. In this conventional skin electrode assembly, the patient-worn assembly typically includes an electrode assembly with one or more electrodes that can be adhered to the patient's skin and an associated connector assembly with an associated number of contacts of the one or more electrodes of the electrode assembly. The connector assembly can be connected to a monitoring-system lead to provide an electrical connection between the one or more electrodes of the electrode assembly and the sensing-and-processing circuitry of the monitoring system.

All patient-worn sensors should be comfortable for the patient. Additionally, the components should be flexible, dimensionally small, chemically inert, resistant to disinfectants, nontoxic, hypo-allergenic to the human body, easy to use, rugged enough to survive impacts from drops, and provide one or more methods for attaching to the patient. In addition to complying with these considerations, conventional patient-worn sensors can have problems with adhesion and electrical contact. That is, patient-worn sensors need to be reliable and maintain contact with the patient throughout all standard use conditions, including, for example, during patient movement. Furthermore, patient-worn sensors need to minimize variations across electrodes caused by patient movement that induces mechanical stress on the electrodes or patient contact points.

In a known two-piece patient-worn sensor that includes a sensor attached to an adapter that is attached to a patient's skin by tacky monitoring electrodes, users or caregivers may need to remove the sensor from the adapter to replace a low battery while leaving the adapter on the patient. Usually, one removes the sensor by gripping it with one hand while holding the adapter with the other hand and pulling the sensor and adapter apart from each other. Hence, the moment the sensor is disengaged from the adapter, one hand of the user could hit the patient's face because of a strong disengagement force required to pull the sensor and adapter apart.

### SUMMARY OF THE INVENTION

To overcome the problems described above, preferred embodiments of the present invention provide patient-monitoring devices that include push tabs on a sensor and an adapter to facilitate disconnecting the sensor and the adapter. In addition, preferred embodiments of the present invention provide patient-monitoring devices that include a flexible printed circuit that includes a rigid portion with a stiffener where electrical components are connected to the flexible printed circuit and that includes a flexible portion without the stiffener to allow the flexible portion to move in two or three dimensions.

According to a preferred embodiment of the present invention, a patient-monitoring device includes an adapter with a flexible printed circuit, the flexible printed circuit includes a rigid portion with a rigid stiffener and a flexible portion with a trace, and with a connector that is connected to one end of the trace, that is movable in two or three dimensions independent of the rigid portion, and that is connectible to an electrode assembly.

The rigid stiffener preferably overlaps with electronic components on the flexible printed circuit. Preferably, the flexible portion extends from the rigid portion and has a meander shape. The flexible portion preferably extends from the rigid portion with a gap between the flexible portion and the rigid portion. The flexible portion preferably extends with a straight portion from the rigid portion. The flexible portion preferably extends with a straight portion from the rigid portion and is terminated in a spiral portion.

The flexible printed circuit preferably includes an additional rigid portion that includes an additional rigid stiffener. Preferably, the patient-monitoring device further preferably includes a sensor assembly that is connectible to and disconnectible from the adapter, a rigid circuit board connected to the additional rigid portion, and a sensor connector connected to the rigid circuit board, wherein the sensor connector is connectible to and disconnectible from the sensor assembly.

The patient-monitoring device further preferably includes an electrode assembly that is attachable to a patient. The connector preferably is rotatable in two or three dimensions independent of the rigid portion.

The patient-monitoring device further preferably includes a sensor assembly that is connectible to and disconnectible from the adapter. The sensor assembly preferably includes a grip to assist in gripping the second electronics assembly during connection and disconnection of the sensor assembly from the adapter. The adapter and the sensor assembly both preferably include a push tab to assist connecting or disconnecting the adapter and the sensor assembly. The push tab of the sensor assembly is preferably laterally offset relative to the push tab of the adapter.

According to a preferred embodiment of the present invention, a patient-monitoring device includes an adapter that includes a first push tab and a sensor assembly that is connected to the adapter and that includes a second push tab. The adapter and the sensor assembly are separated by applying to the first and second push tabs forces in opposite directions.

The first and second push tabs are preferably laterally offset. The first and second push tabs are preferably laterally offset such that a user's left thumb and left index finger can be used to separate the adapter and the sensor assembly. Preferably, one of the adapter and the sensor assembly includes arms, and the other of the sensor assembly and the adapter includes ledges that are engageable with the arms. Each of the arms preferably includes a slot that receives a corresponding ledge. The sensor assembly preferably includes a grip to assist in gripping the sensor assembly.

According to a preferred embodiment of the present invention, a patient-monitoring device includes an adapter including a flexible printed circuit with a rigid portion that includes a rigid stiffener, including a connector that is connectible to an electrode assembly, and including a discrete wire that connects the flexible printed circuit and the connector. The connector is movable in two or three dimensions independent of the rigid portion.

Preferably, the patient-monitoring device further includes the electrode assembly that is attachable to a patient. The connector preferably is rotatable in two or three dimensions independent of the rigid portion.

According to a preferred embodiment of the present invention, a patient-monitoring device includes an adapter with a flexible printed circuit, the flexible printed circuit includes a rigid portion that includes a rigid stiffener and a flexible portion that includes a trace and that extends from the rigid portion with a gap between the flexible portion and the rigid portion, and with a connector that is connected to one end of the trace and connectable to an electrode assembly.

The above and other features, elements, characteristics, steps, and advantages of the present invention will become more apparent from the following detailed description of preferred embodiments of the present invention with reference to the attached drawings.

### BRIEF DESCRIPTION OF THE DRAWINGS

FIG. 1 shows a patient-worn sensor in contact with a patient according to a preferred embodiment of the present invention.

FIG. 2 shows a patient-worn sensor according to a preferred embodiment of the present invention.

FIG. 3 shows a sensor according to a preferred embodiment of the present invention.

FIG. 4 shows an adapter according to a preferred embodiment of the present invention.

FIGS. 5 and 6 respectively show front and rear views of a sensor according to a preferred embodiment of the present invention.

FIGS. 7 and 8 respectively show front and rear perspective views of an adapter according to a preferred embodiment of the present invention.

FIG. 9 shows a partial exploded view of an adapter according to a preferred embodiment of the present invention.

FIG. 10 shows a flexible printed circuit according to a preferred embodiment of the present invention.

FIG. 11 shows a plan view of flexible printed circuit according to a preferred embodiment of the present invention.

FIG. 12-16 show plan views of examples of flexible printed circuits according to preferred embodiments of the present invention.

### DETAILED DESCRIPTION OF PREFERRED EMBODIMENTS

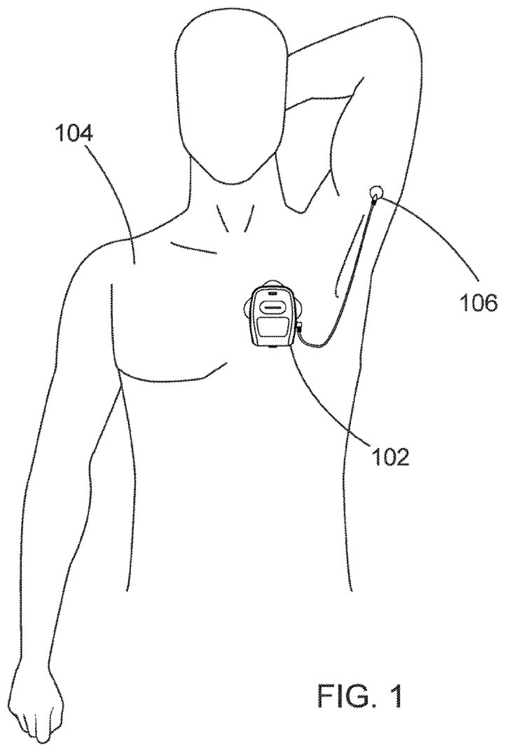

FIG. 1 shows a patient-worn sensor 102 affixed to a patient 104. In the example shown in FIG. 1, the patient-worn sensor 102 is attached to the chest of the patient 104. However, the patient-worn sensor 102 can be affixed to one or more other areas on the patient's 104 body. The patient-worn sensor 102 can detect, record, store, and transmit various vital signs and other information of the patient 104 or of another patient with which the patient-worn sensor 102 is in contact. For example, the patient-worn sensor 102 can include several adherent electrodes that contact the skin of the patient 104 to measure various biological information, vital signs, and patient information including, but not limited to, heart rhythm, heart rate, blood pressure, body temperature, respiratory rate, blood oxygenation, blood glucose level, hydration levels, perspiration, and bio-impedance. The patient-worn sensor 102 can also track patient motion, movement, activity, position, posture, and physical location.

The patient-worn sensor 102 can also communicate with one or more other computing devices, either through wired or wireless communication. For example, the patient-worn sensor 102 can use Bluetooth, WiFi, or a cellular communication protocol to communicate with other computing devices such as bedside monitors, personal computers, tablet devices, mobile phones, central servers, or a cloud-based network. As an example, the patient-worn sensor 102 can transmit vital-sign information collected from the patient 104 to a tablet device or personal computer functioning as a bedside monitor. The tablet or personal computer can process received information and display the information in a readably understandable format to a caretaker or other user. For example, a tablet device can receive vital-sign information from the patient-worn sensor 102 through a Bluetooth connection and display an electrocardiogram (ECG) waveform of the patient as well as information on the patient's heart rate, respiration rate, blood oxygenation level, body temperature, and/or other vital signs. As another example, the patient-worn sensor 102 can be periodically connected to a computing device (such as a bedside monitor) through a wired connection to allow information collected by the patient-worn sensor 102 to be stored, processed, and displayed by the computing device and/or transferred to one or more other computing devices (e.g., personal computers, servers located at the hospital, cloud storage servers, etc.).

Furthermore, information recorded by the patient-worn sensor 102 can be transmitted to other computing devices for use in both real-time or near real-time analysis of the patient 104's condition as well as for tracking of vital-sign information for the patient 104 over time. For example, the information recorded by the patient-worn sensor 102 can be transmitted to a display device to allow caregivers to observe the information and make adjustments to patient care for the patient 104 based on the information. The information can also be transmitted to a central information repository to allow historical vital-sign and other information for the patient to be logged. Both real-time and historical vital-sign information, and other information, for a patient can be accessed by caregivers who are not at the same physical location as the patient. For example, vital-sign information collected by the patient-worn sensor 102 can be transmitted to a mobile device owned by the patient 104 (e.g., a smart phone) to allow the patient 104 to view the information. The information can further be transmitted to a central server that can be accessed by one or more caregivers (e.g., using personal computers or mobile devices) to allow the caregivers to view the collected information and make patient care decisions for the patient 104 from a location that is remote from where the patient 104 is located.

Other components that can be included as part of the patient-worn sensor 102 include a power supply, buttons, or other input mechanisms for receiving user input, one or more audible alarms or speakers, and lights or a display screen. A power supply for the patient-worn sensor 102 can be a battery pack that receives standard disposable batteries, a rechargeable battery, or a removable battery pack that can be replaced with a fully charged battery pack. The patient-worn sensor 102 can further include input mechanisms such as, for example, buttons, keys, or a touch screen. The input mechanisms can allow the patient 104 or a caregiver to adjust settings for the patient-worn sensor 102, perform various tests (such as sensor tests, battery power level tests, etc.) or reset one or more alarms for the patient-worn sensor 102. The input mechanisms can also allow the patient 104 to place a distress call (e.g., to a caregiver or to a hospital alert system) if the patient 104 is in need of assistance.

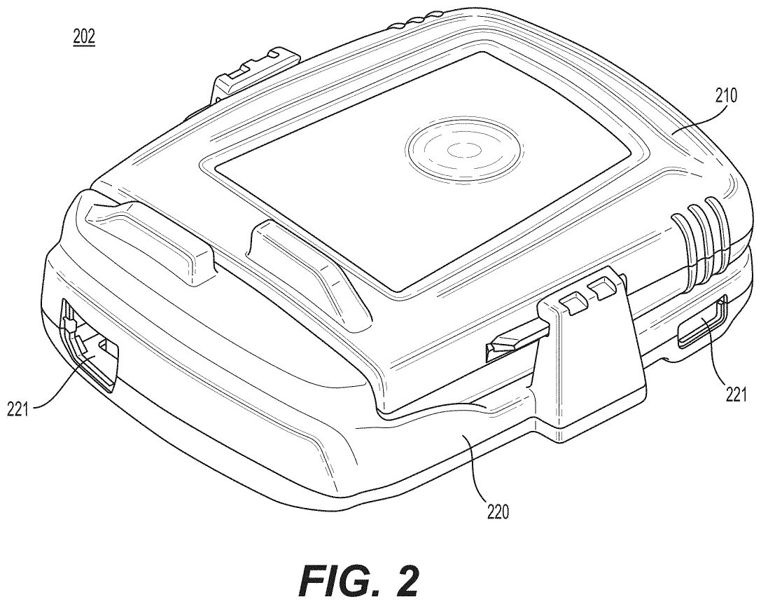

FIG. 2 is a perspective view of an exemplary patient-worn sensor 202. As shown in FIG. 2, the patient-worn sensor 202 can include a sensor 210 coupled to an adapter 220. The adapter 220 can include receptacles 221 that can be connected to different sensors (not shown). The adapter 220 can include any number and any type of receptacles 221.

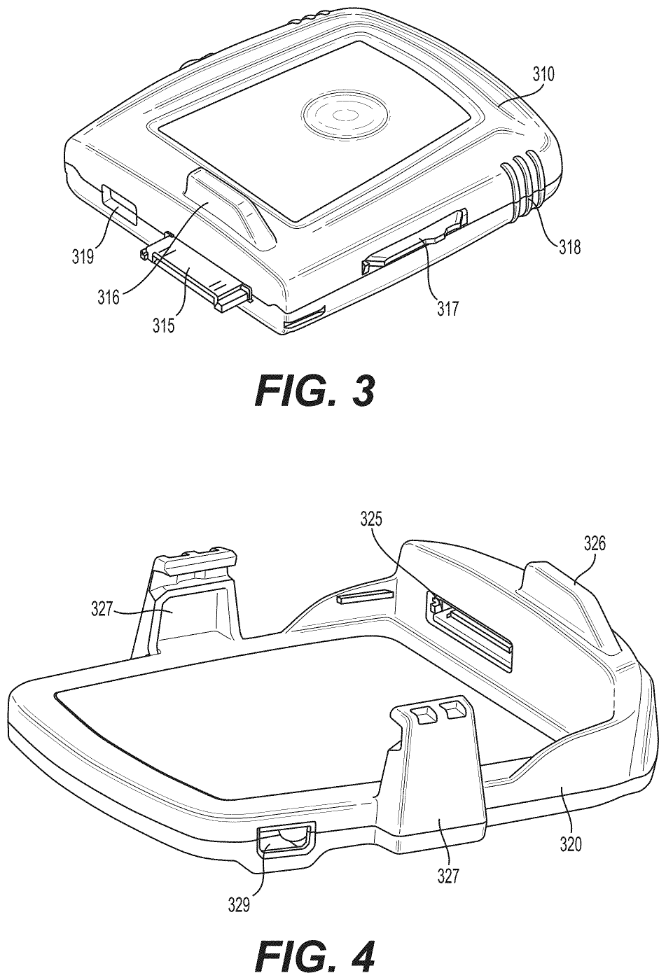

FIGS. 3 and 4 are perspective views of an exemplary sensor 310 and an exemplary adapter 320, respectively. As shown in FIG. 3, the sensor 310 can include an electrical connector 315, a push tab 316, ledges 317, pinch grips 318, and a receptacle 319. The connector 315 plugs into a socket connector 325 of the adapter 320 and is used to transmit and receive power and electrical signals between the sensor 310 and the adapter 320. The push tab 316 of the sensor 310 and the push tab 326 of the adapter 320 can be used to join and separate the sensor 310 and adapter 320. The ledges 317 can be located on opposing sides of the sensor 310 and can engage with corresponding arms 327 of the adapter 320. The ledges 317 and arms 327 can be used to align the sensor 310 with the adapter 320 and to secure the sensor 310 to the adapter 320. The sensor 310 can include pinch grips 318 on opposing sides that allow a user or caregiver to more easily grip the sensor 310. In FIG. 3, the pinch grips 318 include three ridges, but any number and any type of grips can be used. Alternatively, no grips at all can be used. The sensor 310 can include a receptacle 319 that can be used to charge the sensor 310 or to transmit/receive data when the sensor 310 is not connected to the adapter 320. For example, the receptacle 319 can be used to transmit/receive data to an electronic device that is not the adapter 320.

As shown in FIG. 4, the adapter 320 can include a socket connector 325, a push tab 326, arms 327, and a receptacle 329. The socket connector 325 can receive the connector 315. Instead of the socket connector 325 being located on the adapter 320 and the connector 315 being located on the sensor, the socket connector can be located on the sensor 310 and the connector can be located on the adapter 320. The push tab 326 can be used with the push tab 316 to join and disconnect the sensor 310 from the adapter. The arms 327 can be located on opposing sides of the adapter 320 and can be used with the ledges 317 to align and secure the sensor 310. As shown in FIG. 4, the arms 327 can include slots that receive the ledges 317. Any arrangement and type of arms can be used. Instead of the arms 327 being located on the adapter 320 and the ledges 317 being located on the sensor 310, the arms can be located on the sensor 310 and the ledges can be located on the adapter 320 The receptacle 329 can be used to connect another electrical device such as a patient-worn sensor, a finger-worn sensor, a wrist sensor, battery charger, computing device, and the like.

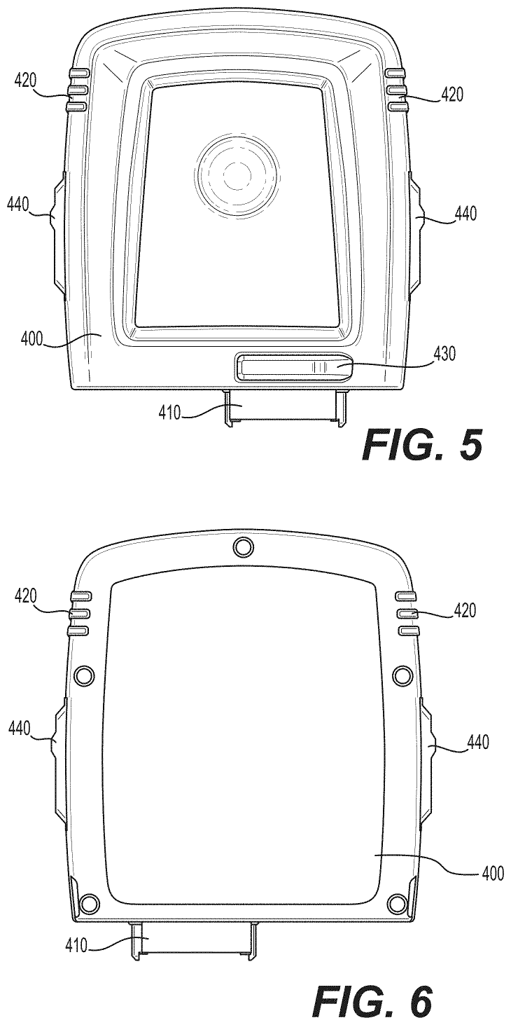

FIGS. 5 is a front view of an exemplary sensor 400 and FIG. 6 is a rear view of the sensor 400. FIGS. 5 and 6 show a connector 410 used to mechanically and electrically connect to an adapter and two pinch grips 420, one on each side of the sensor 400, to assist a user in handling and gripping the sensor 400 when plugging in and unplugging the sensor 400 from an adapter. As shown, the grips 420 are defined by a series of ridges or protrusions in a surface of a housing of the sensor 400, although other suitable features are possible. FIG. 5 also shows a push tab 430 on a front surface of the sensor 400 that can assist a user to apply an insertion or separation force when plugging or unplugging the sensor 400 from an adapter.

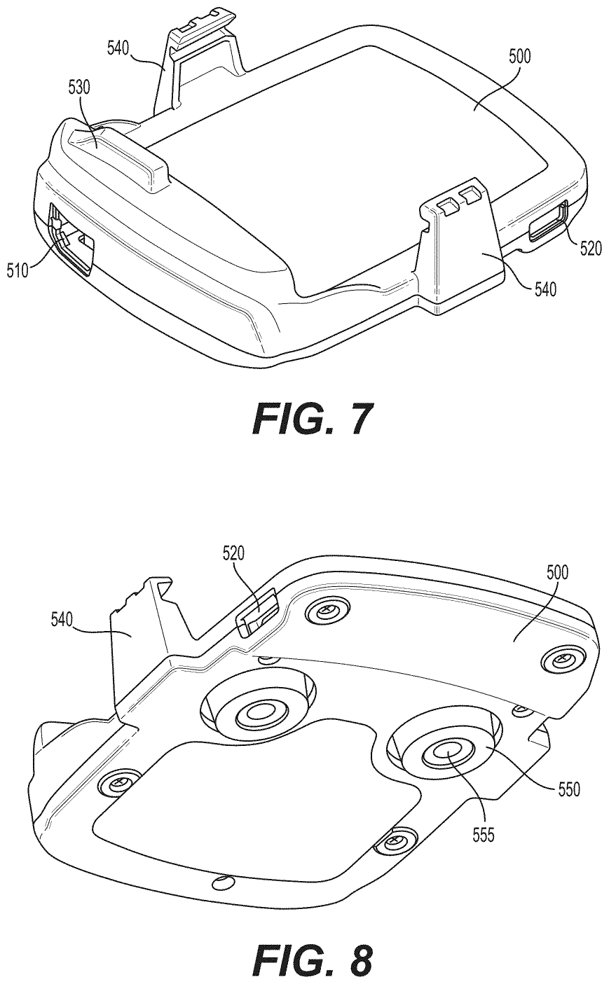

Additionally, FIG. 5 shows two ledges 440, one on each side of the sensor 400, used to interlock with arms on an adapter (shown in FIG. 7) to hold the sensor 400 and the adapter together.

FIGS. 7 and 8 are respective front and rear perspective views of an adapter 500. FIG. 7 shows that the adapter 500 can include additional receptacles 510 and 520 that can be used to access and connect internal electronics via direct wiring or a connector. As shown, the receptacle 510 is on the bottom of the adapter 500, and the receptacle 520 is on the right side, although other locations are possible.

In addition, FIG. 7 shows that the adapter 500 can include two arms 540, one on each side of the adapter 500. As shown, the arms 540 can include a slot and a recess facing inward that assist in aligning and securing a sensor when plugging and unplugging the sensor to and from the adapter 500. The slot and/or the recess in the arms 540 can engage with the ledges on the sides of a sensor to interlock with the ledges to hold the sensor and the adapter 500 together.

FIG. 7 also shows a push tab 530 on a front surface of the adapter 500 that can be used to assist a user to apply an insertion or separation force when plugging or unplugging a sensor from the adapter 500. As shown in FIG. 2, the push tabs on the front surfaces of the sensor 210 and the adapter 220 are laterally offset from each other so that a user can access both sides of the push tabs to use a thumb and a finger to either pull together or push apart the sensor 210 to or from the adapter 200 when plugging or unplugging the two devices.

When a user tries to remove a sensor from an adapter, he or she can put fingers, including thumbs, on the push tabs and push them in opposite directions to easily disengage the sensor. More particularly, a user can put a left thumb on the bottom side of the push tab of the sensor and a left index finger on the top side of push tab of the adapter while holding the sensor grips with a right thumb and a right index finger. Then, by a twisting motion of the left finger and thumb, the push tabs are pushed away from each other so that the sensor is removed from the adapter in a safe manner. To encourage users to grip the sensor with the right hand while handling the push tabs with the left hand, the push tab on the sensor can be located to the left of the push tab on the adapter. Assuming the patient-worn sensor is in contact with the left side of a patient's chest, as is often the case, removing the sensor with the user's right hand makes it less likely the user will hit the patient's face when removing the sensor than if the user's left hand is used.

The rear perspective view of FIG. 8 shows an arm 540 and a receptacle 520 on the adapter 500. Additionally, FIG. 8 shows that the adapter 500 can include two stabilizing boots 550, although other amounts are possible. The stabilizing boots 550 extend from the adapter 500 so that each can surround an electrical contact by way of a snap connector 555. The stabilizing boots 550 can absorb shock to the adapter 500 caused by motion of the patient to provide a more stable signal through the snap connector 555. The stabilizing boots 550 can also absorb motion caused by flexing with the patient's skin and can absorb shock due to motion caused by other movements of the patient, such as walking, running, rolling over, etc. Additionally, the stabilizing boots 550 can also absorb instantaneous mechanical shock to the snap connector 555 and an internally coupled trace caused by snapping and unsnapping a mating snap connector.

The stabilizing boots 550 can absorb shock caused by movement of the patient while still allowing for a degree of compliance in the X, Y, and Z directions and in rotations about the X, Y, and Z directions. The stabilizing boots 550 can provide stability to a connection between the patient-worn sensor, the snap connector 555, and an adhesive pad connected to a patient by providing for a greater degree of movement in a Z-axis direction than in X-axis and Y-axis directions of the patient-worn sensor relative to the patient. The Z-axis can be, for example, a direction that is substantially perpendicular to the patient's skin (and therefore the contact surface of the adhesive electrode pad) while the X-axis and Y-axis directions are substantially parallel to the patient's skin (and therefore the contact surface of the adhesive electrode pad). When in use, an adhesive side of the adhesive electrode pad portion of the patient-worn sensor is affixed to a patient while the opposite side of the adhesive electrode pad is attached to the patient-worn sensor by way of disposable snap connectors of electrodes of the adhesive electrode pad engaging the electrical contacts 555. Once the patient-worn sensor is attached to the patient, the patient is free to move, which can cause shifting in the relative location of contact points on the patient's skin at which the electrodes of the adhesive electrode pad contact the patient. The stabilizing boots 550 can absorb some or all of the motion caused by movement of the patient, thereby reducing negative effects on signal integrity and quality received by the electrodes of the adhesive electrode pad. In some implementations, the stabilizing boots 550 extend beyond the electrical contacts 555 to at least partially encircle portions of the electrodes of the adhesive electrode pad.

The stabilizing boots 550 can be made using an over-molding process, such that the stabilizing boots 550 are integrated with a portion of a housing of the adapter 500 as an integral piece. The over-molding process allows for the stabilizing boots 550 to be made from a material having less rigidity than a material used to form other portions of the adapter housing, while still allowing the stabilizing boots 550 to be defined as an integral piece with at least a portion of the adapter housing. For example, the stabilizing boots 550 can be defined from a thermoplastic elastomer (TPE) material using the over-molding process.

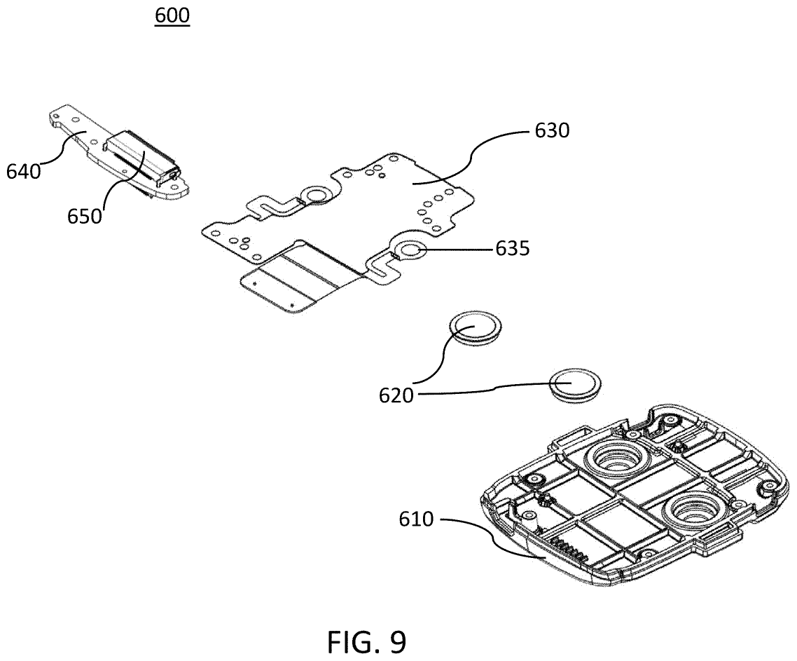

FIG. 9 shows an exploded view of components of an adapter 600 with the top housing removed. Components of the adapter 600 shown in FIG. 9 include a bottom housing 610, snap connectors 620, a flexible printed circuit (FPC) 630, and a connector board 640 that includes a connector 650. As shown, the bottom housing 610 can include structural features used to mount and secure the components of the adapter 600 and the top housing (not shown in FIG. 9).

The top housing and the bottom housing 610 can be made from molded plastic or another material that is suitably light weight so as to facilitate easy attachment to a patient and a sensor, while also structurally strong enough to protect the inner components of the adapter 600 from damage if the adapter 600 or patient-worn sensor is dropped. The top housing and the bottom housing 610 can, for example, be plastic injection molded parts molded from a high impact plastic such as polycarbonate (PC), acrylonitrile butadiene styrene (ABS) or an ABS/PC blend. The top housing and the bottom housing 610 can also engage with each other in a water-tight or semi-water-tight seal to prevent moisture from reaching the inner components of the adapter 600.

As shown in FIG. 9, the bottom housing 610 can include two recesses used to locate and align the two snap connectors 620 that are supported by the stabilizing boots 550 that protrude from the rear of the bottom housing 610, as shown in FIG. 8. The snap connectors 620 can mate with corresponding snap connectors of an electrical device such as a sensor or a disposable electrode connected to the patient to form both a mechanical connection and an electrical connection between the electrical device and the adapter 600. Additionally, each connection of a corresponding mating snap connector to one of the snap connectors 620 forms an electrical connection to transmit and receive signals from the electrical device to the processing components of the FPC 630.

The FPC 630 can include a rigid portion with various processing components implemented as integrated circuit chips and/or other electrical components (not shown). Thus, the FPC 630 can be made from polyimide, or any other suitable material. The FPC 630 can include passive components, such as resistors, and can transmit signals. The FPC 630 can also include active components, such as one or more processors, that can process signals received from a patient, and can include one or more communications modules to communicate with other computing devices using one or more communications protocols (e.g., Bluetooth, WiFi, cellular communication, etc.). Other components that can be included in the circuitry of the FPC 630 are interface circuits to interface with an electronic device, such as a sensor, one or more accelerometers, a GPS or other location detection circuit, temperature sensors to sense environmental temperature, light sensors to sense environmental light, and output circuits to output information to a user through the control of output devices such as lights, speakers, audible alarms, display screens, etc.

The FPC 630 can further include flexible portions 635 that are aligned with each of the snap connectors 620. The flexible portions 635 includes traces that are connected to the snap connectors. Each flexible portion 635 is aligned with a snap connector 620 that receives a mating snap connector of an electrical device, such as a sensor that can be connected to a patient or an electrode that can be connected to a patient. Instead of using flexible portions 635, it is possible to use discrete wires to connect the FPC 630 to the snap connectors 620.

Rather than bending a portion of the FPC 630, for example, by 180°, to allow the FPC 630 to be connected with the sensor, the FPC 630 can use the connector board 640 and connector 650. The connector board 640 can be electrically connected to the FPC 630 directly by soldering, by discrete wiring, or via an additional stacking connector. The connector board 640 is preferably a rigid printed circuit board (PCB) but can be a flexible circuit board or any other suitable substrate.

The flexible portions 635 are used to absorb movement of the snap connectors 620 following any patient skin movement. However, a problem of trace disconnection can occur near mounted electrical components due to high stress concentration that is caused when the FPC 630 is unintentionally bent or mishandled during the assembly process. To prevent the problem of trace fatigue or damage, stiffeners can be added to portions of FPC 630 where electrical components are mounted.

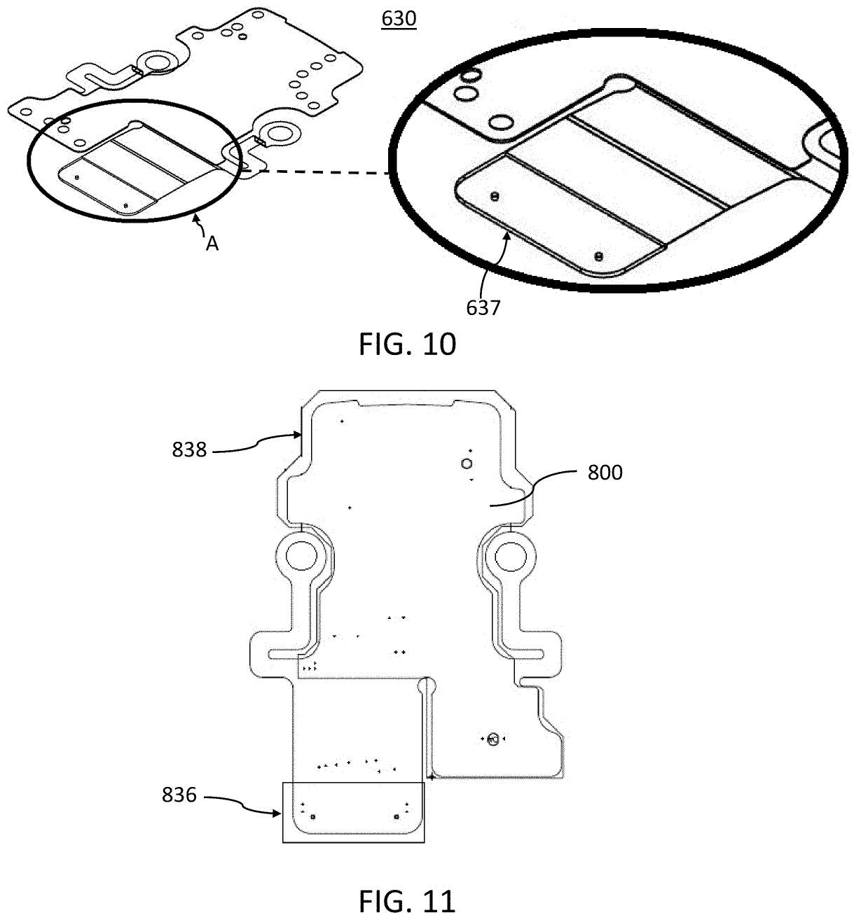

The left-hand portion of FIG. 10 shows the FPC 630 of FIG. 9, and includes a portion of the FPC 630 within circle A that is connected to the connector board 640. The right-hand portion of FIG. 10 shows circle A blown up. As shown in the blown up area, the FPC 630 can include a stiffener 637 adhered to the underside of the FPC 630, overlapping with the area where the connector board 640 is attached. This stiffener 637 is more rigid than the material of the FPC 630 and adds mechanical strength to the portion of the FPC 630 to which it is attached. The added stiffener 637 eliminates or minimizes the problem of trace fatigue or damage during handling and assembly. The stiffener 637 can be made from a glass-reinforced epoxy laminate material such as FR4, G10, or any other suitable material. The stiffener can be about 0.4 mm thick within manufacturing tolerances, or can be any other suitable thickness.

FIG. 11 is a plan view of a FPC 800 similar to that shown in FIGS. 9 and 10. This view shows that stiffeners can be added in outlined areas 836 and 838 that can include electrical components and used to increase mechanical strength and integrity. The stiffener can be about 0.2 mm thick, within manufacturing tolerances, or can be any other suitable thickness.

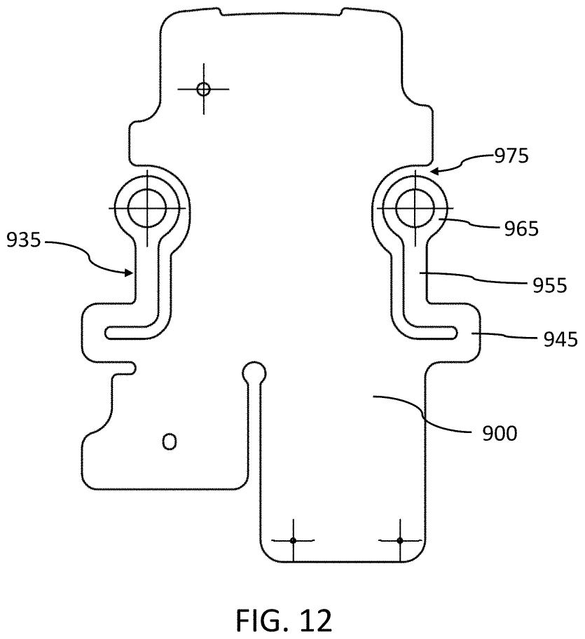

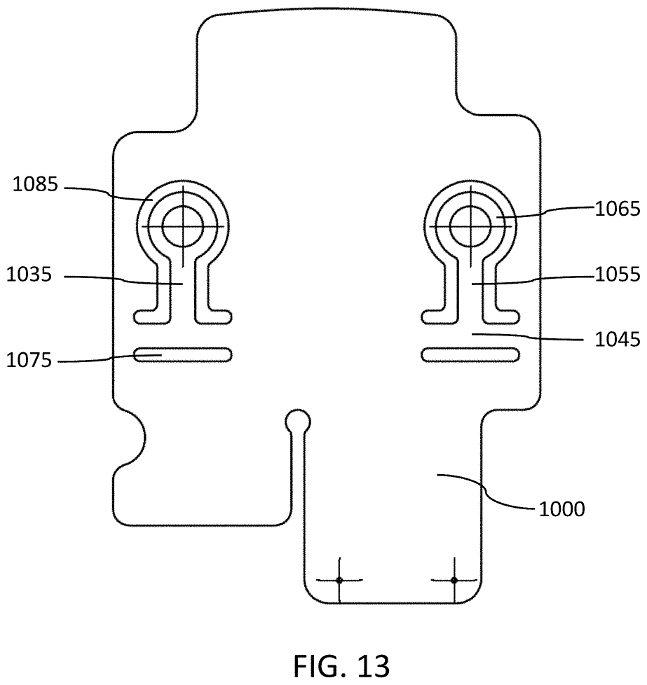

Another problem can arise by trace fatigue in the FPC caused by forces from the stabilizing boots through the snap connectors during repeated movement of the stabilizing boots by patient skin motion or snapping and unsnapping of the mating snap connectors. Trace fatigue can cause intermittent contact or a complete disconnection of the FPC and associated circuitry with the snap connector, the mating snap connector, and the electrical device such as a sensor. This problem can be eliminated or minimized by using different configurations of trace routing in the FPC. FIGS. 12-14 show plan views of examples of FPCs with different possible configurations of trace routing.

FIG. 12 shows a FPC 900 similar to that illustrated in previous figures. The FPC 900 can include a flexible portion 935 in a meander shape, with one trace or electrode that is located on each side the FPC 900 and that connect to corresponding snap connectors. The snap connector is centered to the annular ring 965 and can be connected to the annular ring 965 by soldering an upper half of the annular ring 965 where copper is not covered by a cover layer, which allows more movement of the snap connector, decreasing trace fatigue. Instead of soldering, the snap connector and the annular ring 965 can be connected in any suitable manner.

As shown, the meander shape of a flexible portion 935 extends from the FPC 900 and includes a U-shaped portion 945 closest to the main portion of the FPC 900 that continues to a straight portion 955 and terminates in the annular ring 965. The annular ring 965 is connected to a corresponding snap connector during adapter assembly. As shown, there is a gap 975 between the flexible portion 935 and the main portion of the FPC 900. This gap 975 allows the annular ring 965 to move in three dimensions relative to the main portion of the FPC 900. This allows the flexible portions 935 to absorb mechanical stresses without damage to the traces caused by any movement of the annular ring 965. The meander shape feature can increase compliance of the FPC 900, not only in the Z-axis of snap up and down movement, but also for movement in the X- and Y-axes in a plane perpendicular to the Z-axis. In addition, the meander shape feature allows for rotations around the X, Y, and Z directions. This meander shape also allows displacement of snaps without arising high stress in the FPC. Therefore, the meander-shaped trace can prevent fracture of the FPC.

FIG. 13 shows a FPC 1000 that includes an inverted T-shaped flexible portion 1035. In the plan view of FIG. 13, the inverted T-shaped flexible portion 1035 includes a horizontal portion 1045 separated from the main portion of the FPC 1000. The T-shaped flexible portion 1035 further includes a vertical portion 1055 separated from the main portion of the FPC 1000 that extends from substantially a center of the horizontal portion 1045 and terminates in an annular ring 1065. The annular ring 1065 is connected to a corresponding snap connector during adapter assembly. As shown, there are two gaps 1075 and 1085 between the flexible portion 1035 and the main portion of the FPC 1000. These gaps 1075, 1085 allow the annular ring 1065 to move in three dimensions relative to the main portion of the FPC 1000 and to rotate in three dimensions about the main portion of the FPC 1000. This allows the flexible portions 1035 to absorb mechanical stresses without damage to the traces caused by any movement of the annular ring 1065.

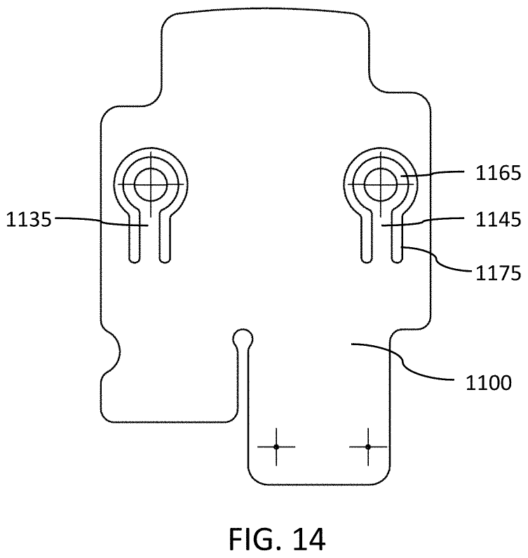

FIG. 14 shows a FPC 1100 that includes a ring-terminal flexible portion 1135. In the plan view of FIG. 14, the ring-terminal flexible portion 1135 includes a vertical portion 1145 separated from the main portion of the FPC 1100 that terminates in an annular ring 1165. The annular ring 1165 is connected to a corresponding snap connector during adapter assembly. As shown, there is a gap 1175 between the flexible portion 1135 and the main portion of the FPC 1100. This gap 1175 allows the annular ring 1165 to move in three dimensions relative to the main portion of the FPC 1100 and to rotate in three dimensions about the main portion of the FPC 1100. This also allows the flexible portions 1135 to absorb mechanical stresses without damage to the traces caused by any movement of the annular ring 1165.

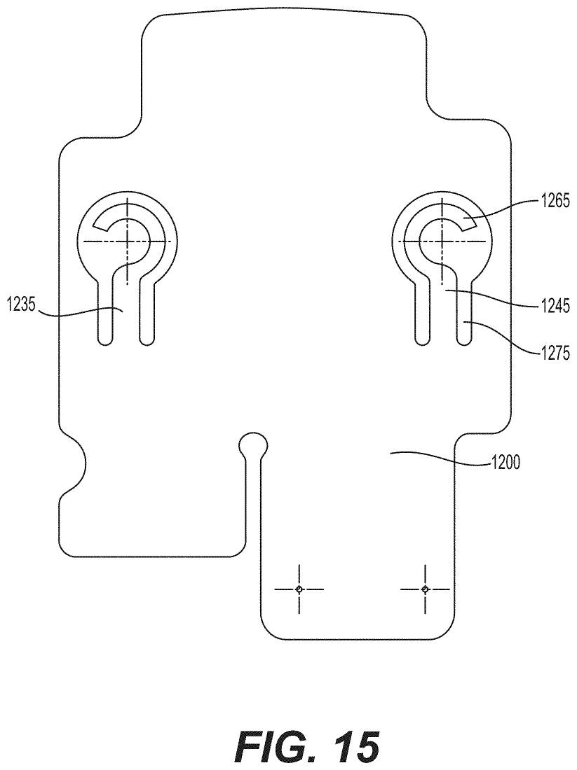

In a modification of the ring-terminal flexible portion, FIG. 15 shows a FPC 1200 that includes a spiral-shaped flexible portion 1235. The spiral-shape is similar to a question mark (or backwards question mark) where the annular ring is not fully defined. In the plan view of FIG. 15, the spiral-shaped flexible portion 1235 includes a vertical portion 1245 separated from the main portion of the FPC 1200 that terminates in a spiral-shaped portion 1265. The spiral-shaped portion 1265 is connected to a corresponding snap connector during adapter assembly. As shown, there is a gap 1275 between the flexible portion 1235 and the main portion of the FPC 1200. The spiral-shaped portion can be soldered to the snap connector at the tip portion of the spiral only, which allows the snap connector to move. This gap 1275 allows the spiral-shaped portion 1265 to move in three dimensions relative to the main portion of the FPC 1200 and to rotate in three dimensions about the main portion of the FPC 1200. This also allows the flexible portions 1235 to absorb mechanical stresses without damage to the traces caused by any movement of the spiral-shaped portion 1265.

Optionally, any of the flexible portions shown in the flexible circuits of FIGS. 12-14 can be terminated in a spiral-shaped portion rather than an annular ring.

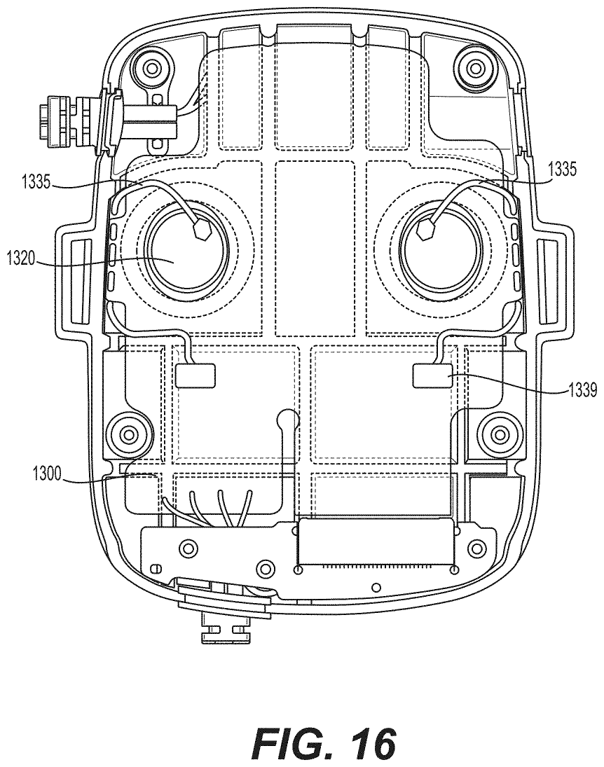

FIG. 16 shows discrete wires 1335 can be used instead of a flexible portion in the FPC 1300. The discrete wires 1335 can either be soldered to the FPC 1300 or can be connected to the FPC 1300 through a connector 1339. The discrete wires 1335 can be soldered to the snap connectors 1320. Alternatively, the discrete wires 1335 can be connected to the FPC 1300 and the snap connectors 1320 is any suitable manner. As shown in FIG. 16, the FPC 1300 can completely surround the snap connector 1320. As shown by the dashed lines in FIG. 16, the discrete wire 1335 can be attached to the snap connector 1320 at a first side of the FPC 1300, can extend under the FPC 1300 to a second side of the FPC 1300 opposite to the first side, and then can extend back to the first side where the discrete wire 1335 attaches to the first side of the FPC 1300. This arrangement of the discrete wire 1335 can prevent the discrete wire 1335 from being pinched between the snap connector 1320 and the top housing when the snap connector 1320 moves up from patient movement or from snapping and unsnapping a mating connector with the snap connector 1320.

Instead of allowing movement in three dimensions, the flexible portion can allow movement in only two dimensions.

It should be understood that the foregoing description is only illustrative of the present invention. Various alternatives and modifications can be devised by those skilled in the art without departing from the present invention. Accordingly, the present invention is intended to embrace all such alternatives, modifications, and variances that fall within the scope of the appended claims.

What is claimed is:

1. : A patient-monitoring device comprising:
an adapter including:
a flexible printed circuit including:
a rigid portion that includes a rigid stiffener; and
a flexible portion that includes a trace; and
a connector that is connected to one end of the trace, that is movable in two or three dimensions independent of the rigid portion, and that is connectible to an electrode assembly.

2. : The patient-monitoring device of claim 1, wherein the rigid stiffener overlaps with electronic components on the flexible printed circuit.

3. : The patient-monitoring device of claim 1, wherein the flexible portion extends from the rigid portion and has a meander shape.

4. : The patient-monitoring device of claim 1, wherein the flexible portion extends from the rigid portion with a gap between the flexible portion and the rigid portion.

5. : The patient-monitoring device of claim 1, wherein the flexible portion extends with a straight portion from the rigid portion.

6. : The patient-monitoring device of claim 1, wherein the flexible portion extends with a straight portion from the rigid portion, and is terminated in a spiral portion.

7. : The patient-monitoring device of claim 1, wherein the flexible printed circuit includes an additional rigid portion that includes an additional rigid stiffener.

8. : The patient-monitoring device of claim 7, further comprising:
a sensor assembly that is connectible to and disconnectible from the adapter;
a rigid circuit board connected to the additional rigid portion; and
a sensor connector connected to the rigid circuit board; wherein
the sensor connector is connectible to and disconnectible from the sensor assembly.

9. : The patient-monitoring device of claim 1, further comprising the electrode assembly; wherein
the electrode assembly is attachable to a patient.

10. : The patient-monitoring device of claim 1, wherein the connector is rotatable in two or three dimensions independent of the rigid portion.

11. : The patient-monitoring device of claim 1, further comprising a sensor assembly that is connectible to and disconnectible from the adapter.

12. : The patient-monitoring device of claim 11, wherein the sensor assembly includes a grip to assist in gripping an electronics assembly during connection and disconnection of the sensor assembly from the adapter.

13. : The patient-monitoring device of claim 11, wherein the adapter and the sensor assembly both include a push tab to assist connecting or disconnecting the adapter and the sensor assembly.

14. : The patient-monitoring device of claim 13, wherein the push tab of the sensor assembly is laterally offset relative to the push tab of the adapter.

15. : A patient-monitoring device comprising:
an adapter that includes a first push tab; and
a sensor assembly that is connected to the adapter and that includes a second push tab; wherein
the adapter and the sensor assembly are separated by applying forces to the first and second push tabs in opposite directions.

16. : The patient-monitoring device of claim 15, wherein the first and second push tabs are laterally offset from each other.

17. : The patient-monitoring device of claim 16, wherein the first and second push tabs are laterally offset from each other such that a user's left thumb and left index finger can be used to separate the adapter and the sensor assembly.

18. : The patient-monitoring device of claim 15, wherein one of the adapter and the sensor assembly includes arms, and the other of the sensor assembly and the adapter includes ledges that are engageable with the arms.

19. : The patient-monitoring device of claim 18, wherein each of the arms includes a slot that receives a corresponding ledge.

20. : The patient-monitoring device of claim 15, wherein the sensor assembly includes a grip to assist in gripping the sensor assembly.

21. : A patient-monitoring device comprising:
an adapter including:
a flexible printed circuit that includes a rigid portion that includes a rigid stiffener;
a connector that is connectible to an electrode assembly; and
a discrete wire that connects the flexible printed circuit and the connector; wherein
the connector is movable in two or three dimensions independent of the rigid portion.

22. : The patient-monitoring device of claim 21, further comprising the electrode assembly; wherein
the electrode assembly is attachable to a patient.

23. : The patient-monitoring device of claim 21, wherein the connector is rotatable in two or three dimensions independent of the rigid portion.

24. : A patient-monitoring device comprising:
an adapter including:
a flexible printed circuit including:
a rigid portion that includes a rigid stiffener; and
a flexible portion that includes a trace and that extends from the rigid portion with a gap between the flexible portion and the rigid portion; and
a connector that is connected to one end of the trace and connectable to an electrode assembly.
